(19) United States
(12) Patent Application Publication
(10) Pub. No.: US 2026/0207858 A1
(43) Pub. Date: Jul. 23, 2026

(54) Safer Medical Needle

(71) Applicant: John Louis Maclaren, Hunters, WA (US)

(72) Inventor: John Louis Maclaren, Hunters, WA (US)

(21) Appl. No.: 18/445,046

(22) Filed: Mar. 20, 2023

(51) Int. Cl. A61M 5/32

(52) U.S. Cl. CPC A61M 5/3286

(57) ABSTRACT

The slanted face of the existing needle design is all one razor edge from top to the bottom.

The lower edge can act like a chisel and peel off some skin which can then be regurgitated into the bloodstream and cause a blood clot or other problems.

The razor point needle is easily damaged but will probably not happen with a solid pointed tip.

A conical needle point will cause less body damage and heal faster.

### Manufacture—Safer Medical Needle

Cut a piece of needle tube as needed.

Compress one end and shape it to a pointed cone.

Add a hub adapter for the syringe to the other end.

The size and length of the cone and slot position may vary.

Stampers, forges, lasers, and other tools may be needed.

Polish as needed.

### BRIEF DESCRIPTION OF THE DRAWINGS

The drawings are enlarged and sectioned for clarity.

FIG. 1 is a top view of the needle, showing the needle hole on the side of the needle instead of the tip of the needle.

FIG. 2 shows the side center section of FIG. 1.

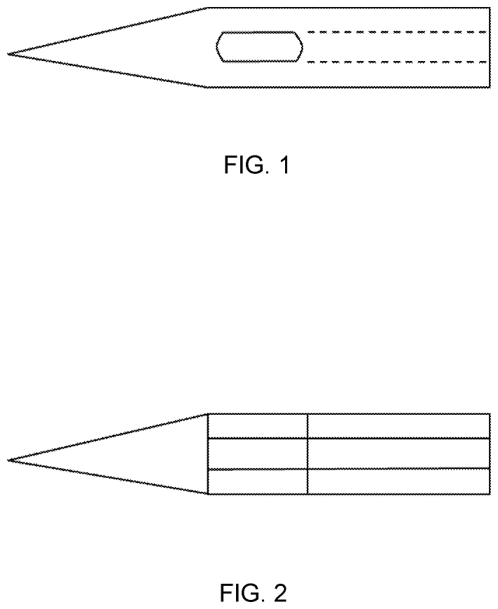

### DESCRIPTION

Installing the needle hole to the side of the needle removes the razor sharp circular cutting edge form the presently used needle. The new needle design does not have a sharp cutting edge which may slice off a piece of skin and be flushed into the bloodstream and cause a blood clot in a major part of the body.

1. The Safer Medical Needle does not create and provide a pathway for skin particles to enter the bloodstream with the possibility of causing infections or blood clots.
There is no razor like incision, therefore quicker healing.
